(19) United States
(12) Patent Application Publication
(10) Pub. No.: US 2021/0285038 A1
(43) Pub. Date: Sep. 16, 2021

(54) Methods of Whole Genome Digital Amplification

(71) Applicant: President and Fellows of Harvard College, Cambridge, MA (US)

(72) Inventors: Xiaoliang Sunney Xie, Lexington, MA (US); Dong Xing, Cambridge, MA (US); Chi-Han Chang, Cambridge, MA (US)

(73) Assignee: President and Fellows of Harvard College, Cambridge, MA (US)

(21) Appl. No.: 16/326,405

(22) PCT Filed: Aug. 31, 2016
(86) PCT No.: PCT/CN16/97520

§ 371 Date: Feb. 19, 2019

(51) Int. Cl. C12Q 1/6853; C12Q 1/6806

(52) U.S. Cl. CPC C12Q 1/6853; C12Q 1/6806

(57) ABSTRACT

The present disclosure provides a method for genomic DNA amplification, such as whole genome amplification, including using a transposase system to make fragments of the genomic DNA including primer binding sites, isolating in oil each fragment within its own aqueous microdroplet along with PCR amplification reagents, amplifying each fragment within its own aqueous microdroplet, demulsifying the microdroplets to obtain the amplicons and sequencing the amplicons.

### STATEMENT OF GOVERNMENT INTERESTS

This invention was made with government support under 5DP1CA186693 from the National Institutes of Health. The Government has certain rights in the invention.

### BACKGROUND

### Field of the Invention

Embodiments of the present invention relate in general to methods and compositions for amplifying trace amount of DNA, such as DNA from a single cell, in order to determine its genetic sequences, particularly the entire genome.

### Description of Related Art

The capability to perform single-cell genome sequencing is important in studies where cell-to-cell variation and population heterogeneity play a key role, such as tumor growth, stem cell reprogramming, embryonic development, etc. Single cell genome sequencing is also important when the cell samples subject to sequencing are precious or rare or in minute amounts. Important to accurate single-cell genome sequencing is the initial amplification of the genomic DNA which can be in minute amounts.

Multiple displacement amplification (MDA) is a common method used in the art with genomic DNA from a single cell prior to sequencing and other analysis. In this method, random primer annealing is followed by extension taking advantage of a DNA polymerase with a strong strand displacement activity. The original genomic DNA from a single cell is amplified exponentially in a cascade-like manner to form hyperbranched DNA structures. Another method of amplifying genomic DNA from a single cell is described in Zong, C., Lu, S., Chapman, A. R., and Xie, X. S. (2012), Genome-wide detection of single-nucleotide and copy-number variations of a single human cell, Science 338, 1622-1626 which describes Multiple Annealing and Looping-Based Amplification Cycles (MALBAC). Another method known in the art is degenerate oligonucleotide primed PCR or DOP-PCR. Several other methods used with single cell genomic DNA include Cheung, V. G. and S. F. Nelson, Whole genome amplification using a degenerate oligonucleotide primer allows hundreds of genotypes to be performed on less than one nanogram of genomic DNA, Proceedings of the National Academy of Sciences of the United States of America, 1996. 93(25): p. 14676-9; Telenius, H., et al., Degenerate oligonucleotide-primed PCR: general amplification of target DNA by a single degenerate primer, Genomics, 1992. 13(3): p. 718-25; Zhang, L., et al., Whole genome amplification from a single cell: implications for genetic analysis. Proceedings of the National Academy of Sciences of the United States of America, 1992, 89(13): p. 5847-51; Lao, K., N. L. Xu, and N. A. Straus, Whole genome amplification using single-primer PCR, Biotechnology Journal, 2008, 3(3): p. 378-82; Dean, F. B., et al., Comprehensive human genome amplification using multiple displacement amplification, Proceedings of the National Academy of Sciences of the United States of America, 2002. 99(8): p. 5261-6; Lage, J. M., et al., Whole genome analysis of genetic alterations in small DNA samples using hyperbranched strand displacement amplification and array-CGH, Genome Research, 2003, 13(2): p. 294-307; Spits, C., et al., Optimization and evaluation of single-cell whole-genome multiple displacement amplification, Human Mutation, 2006, 27(5): p. 496-503; Gole, J., et al., Massively parallel polymerase cloning and genome sequencing of single cells using nanoliter microwells, Nature Biotechnology, 2013. 31(12): p. 1126-32; Jiang, Z., et al., Genome amplification of single sperm using multiple displacement amplification, Nucleic Acids Research, 2005, 33(10): p. e91; Wang, J., et al., Genome-wide Single-Cell Analysis of Recombination Activity and De Novo Mutation Rates in Human Sperm, Cell, 2012. 150(2): p. 402-12; Ni, X., Reproducible copy number variation patterns among single circulating tumor cells of lung cancer patients, PNAS, 2013, 110, 21082-21088; Navin, N., Tumor evolution inferred by single cell sequencing, Nature, 2011, 472 (7341):90-94; Evrony, G. D., et al., Single-neuron sequencing analysis of 11 retrotransposition and somatic mutation in the human brain, Cell, 2012. 151(3): p. 483-96; and McLean, J. S., et al., Genome of the pathogen Porphyromonas gingivalis recovered from a biofilm in a hospital sink using a high-throughput single-cell genomics platform, Genome Research, 2013. 23(5): p. 867-77. Methods directed to aspects of whole genome amplification are reported in WO 2012/166425, U.S. Pat. No. 7,718,403, US 2003/0108870 and U.S. Pat. No. 7,402,386.

However, a need exists for further methods of amplifying small amounts of genomic DNA, such as from a single cell or small group of cells.

### SUMMARY

The present disclosure provides a method for genomic DNA amplification, such as whole genome amplification, such as uniform amplification of genomic DNA, including using a transposase system to make fragments of the genomic DNA including primer binding sites, isolating each fragment within its own aqueous microdroplet along with amplification reagents, amplifying each fragment within its own aqueous microdroplet to create amplicons of the fragment within the microdroplet and collecting and sequencing the amplicons from each microdroplet. According to one aspect, the disclosure provides a method for genomic DNA amplification, such as whole genome amplification, such as uniform amplification of genomic DNA, including using a transposase system to make fragments in aqueous media of the genomic DNA and inserting or attaching a specific PCR primer binding site to each fragment, dividing the aqueous media into a large number of aqueous droplets in oil, each droplet of which contains no more than one DNA fragment along with PCR reagents, amplifying each fragment within the droplet by PCR until saturation within each droplet occurs, demulsification of all of the droplets, i.e. lysing the droplets using a demulsification agent, for example, to recover or collect the amplicons, and sequencing of the amplicons.

According to one aspect, a method is provided for genomic DNA amplification, such as whole genome amplification, including using a transposase system to make fragments of the genomic DNA including primer binding sites, isolating in oil each fragment within its own aqueous microdroplet along with PCR amplification reagents, amplifying each fragment within its own aqueous microdroplet, demulsifying the microdroplets to obtain the amplicons and sequencing the amplicons.

Embodiments of the present disclosure are directed to a method of amplifying DNA such as a small amount of genomic DNA or a limited amount of DNA such as a genomic sequence or genomic sequences obtained from a single cell or a plurality of cells of the same cell type or from a tissue, fluid or blood sample obtained from an individual or a substrate. According to certain aspects of the present disclosure, the methods described herein can be performed in a single tube with a single reaction mixture. According to certain aspects of the present disclosure, the nucleic acid sample can be within an unpurified or unprocessed lysate from a single cell. Nucleic acids to be subjected to the methods disclosed herein need not be purified, such as by column purification, prior to being contacted with the various reagents and under the various conditions as described herein. The methods described herein can provide substantial and uniform coverage of the entire genome of a single cell producing amplified DNA for high-throughput sequencing.

Embodiments of the present invention relate in general to methods and compositions for making DNA fragments, for example, DNA fragments from the whole genome of a single cell which may then be subjected to amplification methods known to those of skill in the art. According to one aspect, a transposase as part of a transposome is used to create a set of double stranded genomic DNA fragments. Each double stranded genomic DNA fragment is isolated in a droplet, such as a microdroplet, along with reagents used to amplify the double stranded genomic DNA fragment. The double stranded genomic DNA fragment is amplified within the droplet, for example, using methods known to those of skill in the art, such as PCR amplification, and as described herein. Accordingly, a method is provided where each double stranded genomic DNA fragment is isolated in a corresponding droplet and is then amplified to produce amplicons. According to one aspect, each fragment within the droplet is amplified to a point where the amplification reactions are saturated. Since each double stranded genomic DNA fragment is isolated and amplified to saturation, the method reduces or eliminates amplification bias which can result when a plurality of double stranded genomic DNA fragments are otherwise amplified within the same reaction volume.

According to certain aspects, methods of making nucleic acid fragments described herein utilize a transposase. The transposase is complexed with a transposon DNA including a double stranded transposase binding site and a first nucleic acid sequence including one or more of a barcode sequence and a priming site to form a transposase/transposon DNA complex. The barcode sequence includes a nucleic acid sequence which uniquely identifies a single cell or group of cells.

The first nucleic acid sequence may be in the form of a single stranded extension. According to certain aspects, the transposases have the capability to bind to the transposon DNA and dimerize when contacted together, such as when being placed within a reaction vessel or reaction volume, forming a transposase/transposon DNA complex dimer called a transposome.

According to one aspect, each transposome includes two transposases and two transposon DNA. The transposon DNA includes a transposase binding site, an optional barcode and a primer binding site. According to one aspect, the transposon DNA includes a single transposase binding site, an optional barcode and a primer binding site. Each transposon DNA is a separate nucleic acid bound to a transposase at the transposase binding site. The transposome is a dimer of two separate transposases each bound to its own transposon DNA. According to one aspect, the transposome includes two separate and individual transposon DNA, each bound to its own corresponding transposase. According to one aspect, the transposome includes only two transposases and only two transposon DNA. According to one aspect, the two transposon DNA as part of the transposome are separate, individual or non-linked transposon DNA, each bound to its own corresponding transposase. As an example, separate and individual transposon DNA as described herein have a single transposon binding site, an optional barcode and a primer binding site.

The transposomes have the capability to randomly bind to target locations along double stranded nucleic acids, such as double stranded genomic DNA, forming a complex including the transposome and the double stranded genomic DNA. The transposases in the transposome cleave the double stranded genomic DNA, with one transposase cleaving the upper strand and one transposase cleaving the lower strand. The transposon DNA in the transposome is attached to the double stranded genomic DNA at the cut site. Accordingly, transposomes are used for transposition, i.e. insertion of the transposon DNA and production of fragments. According to certain aspects, a plurality of transposase/transposon DNA complex dimers bind to a corresponding plurality of target locations along a double stranded genomic DNA, for example, and then cleave the double stranded genomic DNA into a plurality of double stranded fragments with each fragment having transposon DNA attached at each end of the double stranded fragment. According to one aspect, the transposon DNA is attached to or inserted into the double stranded genomic DNA and a single stranded gap exists between one strand of the genomic DNA and one strand of the transposon DNA. According to one aspect, gap extension is carried out to fill the gap and create a double stranded connection between the double stranded genomic DNA and the double stranded transposon DNA. According to one aspect, the transposase binding site of the transposon DNA is attached at each end of the double stranded fragment. According to certain aspects, the transposase is attached to the transposon DNA which is attached at each end of the double stranded fragment. According to one aspect, the transposases are removed from the transposon DNA which is attached at each end of the double stranded genomic DNA fragments.

According to one aspect of the present disclosure, the double stranded genomic DNA fragments produced by the transposases which have the transposon DNA attached at each end of the double stranded genomic DNA fragments are then gap filled and extended using the transposon DNA as a template. Accordingly, a double stranded nucleic acid extension product is produced which includes the double stranded genomic DNA fragment and a double stranded transposon DNA including a primer binding site at each end of the double stranded genomic DNA. According to one aspect, the primer binding sites at each end of the double stranded genomic DNA have the same sequence.

The double stranded nucleic acid extension products including the genomic DNA fragment are then isolated in droplets, such as microdroplets as can be produced by an emulsion droplet technique known to those of skill in the art or mixing an oil phase with an aqueous phase for creation of microdroplets spontaneously or otherwise, along with amplification reagents known to those of skill in the art. According to one aspect, each droplet includes one double stranded nucleic acid extension product, i.e. one double stranded genomic nucleic acid fragment with associated primer binding sites, and amplification reagents and the double stranded genomic nucleic acid fragment may then be amplified using methods known to those of skill in the art, such as PCR, to produce amplicons of the double stranded genomic nucleic acid fragment. According to one aspect, the amplicons from each droplet are released, such as by droplet lysis and collected for further analysis such as sequencing using methods known to those of skill in the art to identify the fragment sequence and the associated barcode sequence, if desired. The collected amplicons may be purified prior to further analysis.

Embodiments of the present disclosure are directed to a method of amplifying DNA using the methods described herein such as a small amount of genomic DNA or a limited amount of DNA such as a genomic sequence or genomic sequences obtained from a single cell or a plurality of cells of the same cell type or from a tissue, fluid or blood sample obtained from an individual or a substrate. According to certain aspects of the present disclosure, the methods described herein can be performed in a single tube to create the fragments which are then isolated within microdroplets and amplified within the microdroplets with the amplicons being collected from the microdroplets. The term droplet or microdroplet may be used interchangeably herein. The methods described herein avoid, inhibit, prevent, or reduce amplification bias associated with prior art amplification methods where many fragments are amplified together within the same reaction mixture. The methods described herein can provide substantial coverage of the entire genome of a single cell producing amplified DNA for high-throughput sequencing.

According to an additional aspect, methods are provided herein for performing whole genome amplification of single cells with high fidelity and amplification uniformity or coverage across different loci in the genome which is useful for further sequencing or analysis using high throughput sequencing platforms known to those of skill in the art. More uniform whole genome amplification normally leads to higher whole genome coverage. Coverage represents the percentage of a single cell genomic DNA that can be preserved after amplification. For example, 50% coverage means half of the genetic materials have been lost during the process of single cell whole genome amplification. Methods provided herein minimize loss and amplification bias and provide substantially complete or complete genome coverage of DNA sequencing of genomic DNA from a single cell. Methods described herein can amplify greater than 90 percent, greater than 95 percent, greater than 96 percent, greater than 97 percent, greater than 98 percent, or greater than 99 percent of genomic DNA from a single cell while greater than 70 percent or 75 percent of the genomic DNA can be sequenced with a sequencing depth of 7× or 10× or 15× or 30× with little, substantially few or no chimera sequences.

Aspects of the methods of the present disclosure improve allele drop-out rate (ADO). The human genome is a diploid genome, which means there are two copies of each of the 23 chromosomes, one maternal copy and one paternal copy for each chromosome. ADO arises from uneven amplification of the maternal copy and the paternal copy. If a human single cell has a heterozygous mutation, the lack of amplification in one of the two alleles causes ADO, which is the primary cause of false negatives of single cell SNV calling. The ADO is measured by the ratio of the undetected and the actual heterozygous SNVs in a single cell. The methods described herein of in vitro transposition and emulsion droplet amplification reduce allele drop-out rate.

Methods described herein reduce or eliminate creation of sequencing artifacts and facilitate advanced genomic analysis of single cell single nucleotide polymorphisms, copy number variations and structural variations. Methods described herein have particular application in biological systems or tissue samples characterized by highly heterogeneous cell populations such as tumor and neural masses. Methods described herein to amplify genomic DNA facilitate the analysis of such amplified DNA using next generation sequencing techniques known to those of skill in the art and described herein.

The DNA amplification methods of the present disclosure will be useful for amplifying small or limited amounts of DNA, which will allow multiple sites in the DNA sample to be genotyped for high-throughput screening. Additionally, the present method will allow for the rapid construction of band specific painting probes for any chromosomal region, and can also be used to micro dissect and amplify unidentifiable chromosomal regions or marker chromosomes in abnormal karyotypes. The presently disclosed method will also allow for the rapid cloning of amplified DNA for sequencing or generating DNA libraries. Thus, the method will not only be a valuable tool for genotype analysis and high-throughput screening, it should also be a valuable tool in cytogenetic diagnosis. The methods described herein can utilize varied sources of DNA materials, including genetically heterogeneous tissues (e.g. cancers), rare and precious samples (e.g. embryonic stem cells), and non-dividing cells (e.g. neurons) and the like, as well as, sequencing platforms and genotyping methods known to those of skill in the art.

Further features and advantages of certain embodiments of the present disclosure will become more fully apparent in the following description of the embodiments and drawings thereof, and from the claims.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a schematic of transposome formation, genomic DNA fragmentation and transposon insertion, microdroplet formation where each microdroplet includes one genomic DNA fragment and amplification reagents and amplification within each droplet to produce amplicons.

FIG. 2 depicts in schematic a structure of a transposon DNA with a 5′ extension being linear and with or without a barcode, where T is the double stranded transposase binding site, P is a priming site at one end of the extension and B is a barcode sequence.

FIG. 3 is a schematic of one embodiment of a transposon DNA and transposome formation.

FIG. 4 is a schematic of transposome binding to genomic DNA, cutting into fragments and addition or insertion of transposon DNA.

FIG. 5 is a schematic of transposase removal, gap filling and extension to form nucleic acid extension products including genomic DNA.

FIG. 6 is a graph showing DNA fragment size distribution resulting from a transposome fragmentation method and amplification of each individual fragment within a microdroplet, the method of which may be referred to herein as “DIANTI”.

FIG. 7 is a graph of data of sequencing read depth of three single human cells amplified using a transposome fragmentation method and amplification of each individual fragment within a microdroplet.

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The present invention is based in part on the discovery of methods for making DNA fragment templates, such as from genomic DNA, using a transposase or transposome, isolating each DNA fragment template within a corresponding microdroplet, i.e. only one DNA fragment within a microdroplet, and amplifying each DNA fragment template within the corresponding microdroplet, i.e. in the absence of other DNA fragment templates, to produce amplicons. According to one aspect, a microdroplet includes only one DNA fragment template for amplification. The amplicons of each DNA fragment template may be collected from the droplets and sequenced. The collected amplicons form a library of amplicons of the fragments of the genomic DNA.

According to one aspect, a genomic DNA, such as genomic nucleic acid obtained from a lysed single cell, is obtained. A plurality of transposomes, each being a dimer of a transposase bound to a transposon DNA with the transposon DNA having a transposase binding site and a specific primer binding site, are used to cut the genomic DNA into double stranded fragments where the transposon DNA becomes attached to the upper and lower of strands of each double stranded fragment. The specific primer binding site is “specific” insofar as the primer binding site sequence is the same so that only a single primer sequence is needed to amplify each fragment. The double stranded DNA fragments having primer binding sites attached thereto are then processed to fill gaps and loaded into microdroplets along with amplification reagents, with one DNA fragment per droplet, using a microfluidic device having a droplet formation region.

According to one aspect, the number of droplets created exceeds the number of DNA fragments such that only one DNA fragment is isolated in a single droplet. Methods of creating droplets of an aqueous phase are known to those of skill in the art. This aspect of the disclosure eliminates competition between DNA fragments during amplification as each droplet isolates a single DNA fragment for amplification within the droplet. Specific primers targeting the transposon binding site and the priming site are used with a DNA polymerase to amplify each fragment which in total equals the whole genomic DNA. After amplification, the droplets are lysed and the amplification products are collected for further analysis.

According to one aspect, the combination of the transposon system and the microdroplet amplification method results in even amplification of the genomic DNA, i.e. the whole genome obtained from a single cell, for example. In vitro transposition is used to add specific priming sites to genomic DNA fragments to avoid using degenerate oligonucleotides. In this aspect, the same primer sequence is used to amplify each fragment of the whole genome. An exemplary method uses microdroplets to physically separate single cell genomic DNA fragments before amplification thereby eliminating competition among different fragments during amplification which results in uniform whole genome amplification.

As indicated, DNA fragment templates made using the transposase methods described herein can be amplified within microdroplets using methods known to those of skill in the art. Microdroplets may be formed as an emulsion of an oil phase and an aqueous phase. An emulsion may include aqueous droplets or isolated aqueous volumes within a continuous oil phase Emulsion whole genome amplification methods are described using small volume aqueous droplets in oil to isolate each fragment for uniform amplification of a single cell's genome. By distributing each fragment into its own droplet or isolated aqueous reaction volume, each droplet is allowed to reach saturation of DNA amplification. The amplicons within each droplet are then merged by demulsification resulting in an even amplification of all of the fragments of the whole genome of the single cell.

In certain aspects, amplification is achieved using PCR. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). The term “polymerase chain reaction” (“PCR”) of Mullis (U.S. Pat. Nos. 4,683,195, 4,683,202, and 4,965,188) refers to a method for increasing the concentration of a segment of a target sequence without cloning or purification. This process for amplifying the target sequence includes providing oligonucleotide primers with the desired target sequence and amplification reagents, followed by a precise sequence of thermal cycling in the presence of a polymerase (e.g., DNA polymerase). The primers are complementary to their respective strands (“primer binding sequences”) of the double stranded target sequence. To effect amplification, the double stranded target sequence is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”) and the target sequence is said to be “PCR amplified.” The PCR amplification reaches saturation when the double stranded DNA amplification product accumulates to a certain amount that the activity of DNA polymerase is inhibited. Once saturated, the PCR amplification reaches a plateau where the amplification product does not increase with more PCR cycles.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself within each microdroplet are, themselves, efficient templates for subsequent PCR amplifications. Methods and kits for performing PCR are well known in the art. All processes of producing replicate copies of a polynucleotide, such as PCR or gene cloning, are collectively referred to herein as replication. A primer can also be used as a probe in hybridization reactions, such as Southern or Northern blot analyses.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reactions are commercially available. Primers useful to amplify sequences from a particular gene region are preferably complementary to, and hybridize specifically to sequences in the target region or in its flanking regions and can be prepared using methods known to those of skill in the art. Nucleic acid sequences generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences. According to one aspect of the present disclosure, each microdroplet includes PCR product of a single template DNA fragment.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

For emulsion PCR, an emulsion PCR reaction is created by vigorously shaking or stirring a “water in oil” mix to generate millions of micron-sized aqueous compartments. Microfluidic chips may be equipped with a device to create an emulsion by shaking or stirring an oil phase and a water phase. Alternatively, aqueous droplets may be spontaneously formed by combining a certain oil with an aqueous phase or introducing an aqueous phase into an oil phase. The DNA library to be amplified is mixed in a limiting dilution prior to emulsification. The combination of compartment size, i.e. microdroplet size, and amount of microdroplets created limiting dilution of the DNA fragment library to be amplified is used to generate compartments containing, on average, just one DNA molecule. Depending on the size of the aqueous compartments generated during the microdroplet formation or emulsification step, up to 3×10⁹ individual PCR reactions per μl can be conducted simultaneously in the same tube. Essentially each little aqueous compartment microdroplet in the emulsion forms a micro PCR reactor. The average size of a compartment in an emulsion ranges from sub-micron in diameter to over a 100 microns, or from 1 picoliter to 1000 picoliters or from 1 nanoliter to 1000 nanoliters or from 1 picoliter to 1 nanoliter or from 1 picoliter to 1000 nanoliters depending on the emulsification conditions.

Other amplification methods, as described in British Patent Application No. GB 2,202,328, and in PCT Patent Application No. PCT/US89/01025, each incorporated herein by reference, may be used in accordance with the present disclosure. In the former application, “modified” primers are used in a PCR-like template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other suitable amplification methods include “race and “one-sided PCR.”. (Frohman, In: PCR Protocols: A Guide To Methods And Applications, Academic Press, N.Y., 1990, each herein incorporated by reference). Methods based on ligation of two (or more) oligonucleotides in the presence of nucleic acid having the sequence of the resulting “di-oligonucleotide,” thereby amplifying the di-oligonucleotide, also may be used to amplify DNA in accordance with the present disclosure (Wu et al., Genomics 4:560-569, 1989, incorporated herein by reference).

According to certain aspects, an exemplary transposon system includes Tn5 transposase, Mu transposase, Tn7 transposase or IS5 transposase and the like. Other useful transposon systems are known to those of skill in the art and include Tn3 transposon system (see Maekawa, T., Yanagihara, K., and Ohtsubo, E. (1996), A cell-free system of Tn3 transposition and transposition immunity, Genes Cells 1, 1007-1016), Tn7 transposon system (see Craig, N. L. (1991), Tn7: a target site-specific transposon, Mol. Microbiol. 5, 2569-2573), Tn10 tranposon system (see Chalmers, R., Sewitz, S., Lipkow, K., and Crellin, P. (2000), Complete nucleotide sequence of Tn10, J. Bacteriol 182, 2970-2972), Piggybac transposon system (see Li, X., Burnight, E. R., Cooney, A. L., Malani, N., Brady, T., Sander, J. D., Staber, J., Wheelan, S. J., Joung, J. K., McCray, P. B., Jr., et al. (2013), PiggyBac transposase tools for genome engineering, Proc. Natl. Acad. Sci. USA 110, E2279-2287), Sleeping beauty transposon system (see Ivics, Z., Hackett, P. B., Plasterk, R. H., and Izsvak, Z. (1997), Molecular reconstruction of Sleeping Beauty, a Tc1-like transposon from fish, and its transposition in human cells, Cell 91, 501-510), To12 transposon system (seeKawakami, K. (2007), To12: a versatile gene transfer vector in vertebrates, Genome Biol. 8 Suppl. 1, S7.)

DNA to be amplified may be obtained from a single cell or a small population of cells. Methods described herein allow DNA to be amplified from any species or organism in a reaction mixture, such as a single reaction mixture carried out in a single reaction vessel. In one aspect, methods described herein include sequence independent amplification of DNA from any source including but not limited to human, animal, plant, yeast, viral, eukaryotic and prokaryotic DNA.

According to one aspect, a method of single cell whole genome amplification and sequencing is provided which includes contacting double stranded genomic DNA from a single cell with Tn5 transposases each bound to a transposon DNA, wherein the transposon DNA includes a double-stranded 19 bp transposase (Tnp) binding site and a first nucleic acid sequence including one or more of an optional barcode sequence and a primer binding site to form a transposase/transposon DNA complex dimer called a transposome. The first nucleic acid sequence may be in the form of a single stranded extension. According to one aspect, the first nucleic acid sequence may be an overhang, such as a 5′ overhang, wherein the overhang includes an optional barcode region and a priming site. The overhang can be of any length suitable to include one or more of an optional barcode region and a priming site as desired. The transposome bind to target locations along the double stranded genomic DNA and cleave the double stranded genomic DNA into a plurality of double stranded fragments, with each double stranded fragment having a first complex attached to an upper strand by the Tnp binding site and a second complex attached to a lower strand by the Tnp binding site. The transposon binding site is attached to each 5′ end of the double stranded fragment. According to one aspect, the Tn5 transposases are removed from the complex. The double stranded fragments are extended along the transposon DNA to make a double stranded extension product having primer binding sites, preferably at each end. According to one aspect, a gap which may result from attachment of the Tn5 transposase binding site to the double stranded genomic DNA fragment may be filled. The double stranded extension product is placed within a droplet amplification reaction volume, such as a microdroplet, along with amplification reagents, and the double stranded genomic DNA fragment is amplified within the droplet. The amplicons, which may include a barcode sequence uniquely identifying the cell or sample from which the double stranded genomic DNA fragment was obtained, are collected, such as by lysis of the droplet. The double stranded DNA amplicons from each droplet may then be sequenced using, for example, high-throughput sequencing methods known to those of skill in the art.

In a particular aspect, embodiments are directed to methods for the amplification of substantially the entire genome without loss of representation of specific sites (herein defined as “whole genome amplification”). In a specific embodiment, whole genome amplification comprises amplification of substantially all fragments or all fragments of a genomic library. In a further specific embodiment, “substantially entire” or “substantially all” refers to about 80%, about 85%, about 90%, about 95%, about 97%, or about 99% of all sequences in a genome.

According to one aspect, the DNA sample is genomic DNA, micro dissected chromosome DNA, yeast artificial chromosome (YAC) DNA, cosmid DNA, phage DNA, P1 derived artificial chromosome (PAC) DNA, or bacterial artificial chromosome (BAC) DNA. In another preferred embodiment, the DNA sample is mammalian DNA, plant DNA, yeast DNA, viral DNA, or prokaryotic DNA. In yet another preferred embodiment, the DNA sample is obtained from a human, bovine, porcine, ovine, equine, rodent, avian, fish, shrimp, plant, yeast, virus, or bacteria. Preferably the DNA sample is genomic DNA.

According to certain exemplary aspects, a transposition system is used to make nucleic acid fragments for amplification and sequencing as desired. According to one aspect, a transposition system is used to fragment genomic DNA into double stranded genomic DNA fragments with the transposon DNA inserted therein. According to one particular aspect, a transposition system is combined with a microdroplet amplification method for single cell genome amplification, where each fragment of a library of DNA fragments created by the transposition system is isolated within a single droplet of an emulsion of aqueous droplets within a oil phase and is then amplified within each droplet, such as by PCR. According to certain aspects, the use of a droplet to make amplicons of a single DNA fragment to the exclusion of other DNA fragments, i.e. in the absence of other DNA fragments of a DNA fragment library, i.e. wherein the droplet includes only one DNA fragment, advantageously achieves high quality amplification of the single-cell genomic DNA (gDNA) reducing or avoiding amplification bias, leading to the noisy single-cell sequencing data that further affect the genome coverage, as well as the low resolution detection of copy number variations (CNVs). PCR by definition is an exponential amplification method, i.e. new copies are made based on the copies from the previous rounds of amplification. According to one aspect, since each DNA fragment of the library of DNA fragments is amplified alone and outside of the presence of other members of the library, little or no amplification bias results because there is little or no slight amplification efficiency difference between amplicons that otherwise may accumulate, and lead to amplification bias between different amplicons after many cycles. According to one aspect where amplification bias efficiency may result as between amplicons, a sufficient number of PCR cycles is used to push the amplification reaction within each droplet to saturation. Once the PCR reactions reach saturation, the different amplicons from different droplets are amplified to a similar amount.

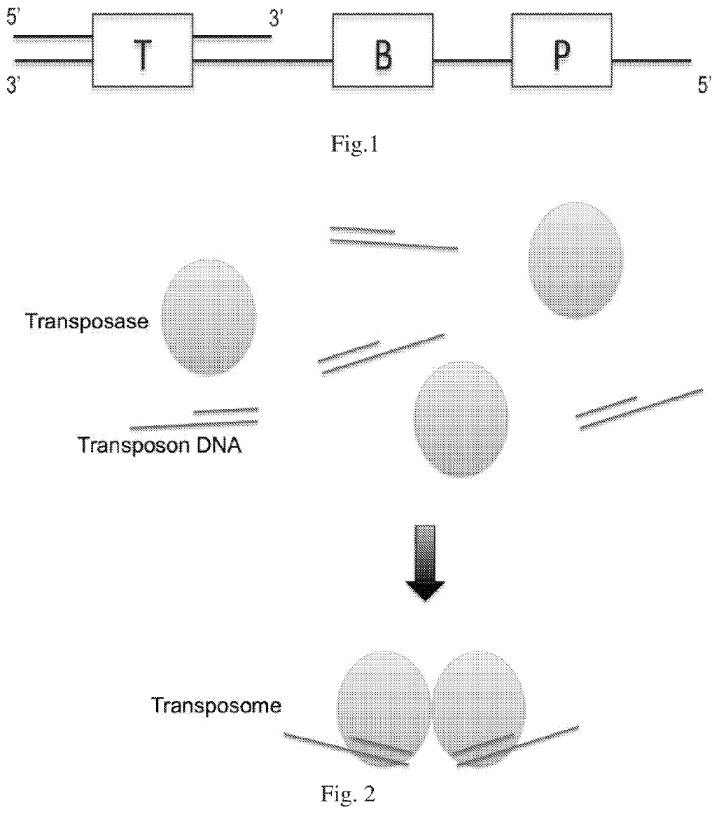

As illustrated in FIG. 1, transposon DNA containing a specific priming site in RED are inserted into the genomic DNA of a single cell while creating millions of small fragments using a transposase. After transposase removal and gap fill-in, the genomic DNA fragments having primer binding sites are loaded into microdroplets. The number of droplets exceeds the number of fragments to ensure that most of the droplets contain only one DNA fragment. Specific primers are then used together with a DNA polymerase to PCR amplify the whole genome of the single cell.

According to certain aspects when amplifying small amounts of DNA such as DNA from a single cell, a DNA column purification step is not carried out so as to maximize the small amount (˜6 pg) of genomic DNA that can be obtained from within a single cell prior to amplification. The DNA can be amplified directly from a cell lysate or other impure condition. Accordingly, the DNA sample may be impure, unpurified, or not isolated. Accordingly, aspects of the present method allow one to maximize genomic DNA for amplification and reduce loss due to purification. According to an additional aspect, methods described herein may utilize amplification methods other than PCR, which is useful in an emulsion droplet amplification method.

According to one aspect and as illustrated in FIG. 2, transposon DNA is designed to contain a double-stranded 19 bp Tn5 transposase (Tnp) binding site at one end, linked or connected, such as by covalent bond, to a single-stranded overhang including an optional barcode region and a priming site at one end of the overhang. Upon transposition, the Tnp and the transposon DNA bind to each other and dimerize to form transposomes.

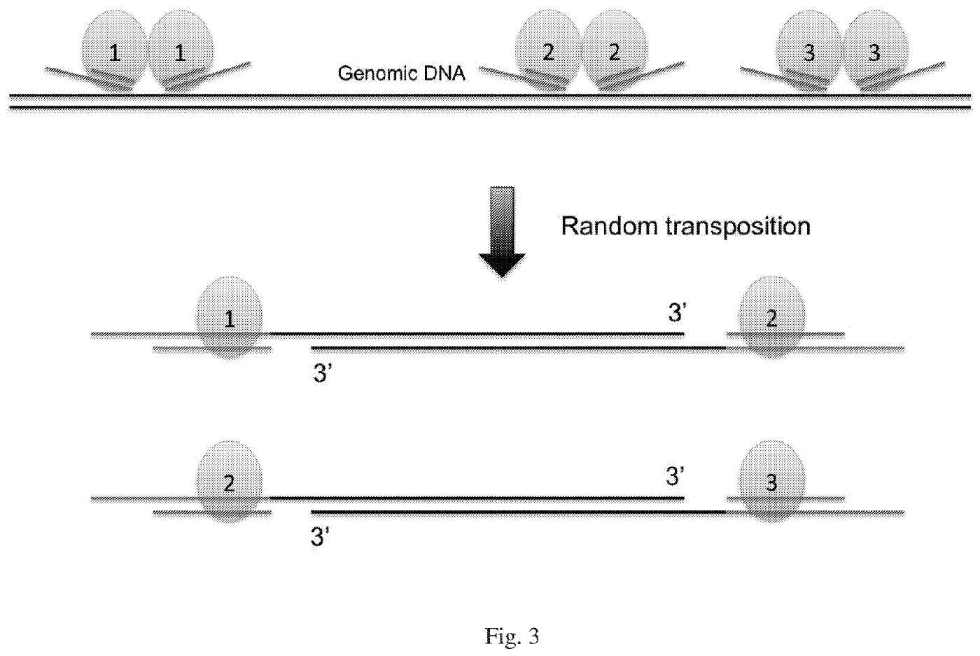

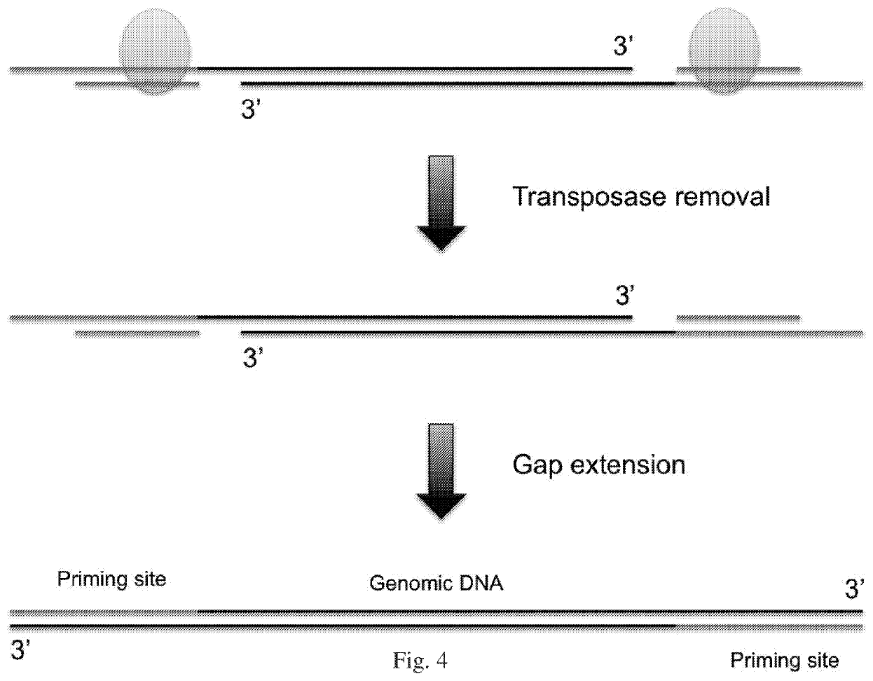

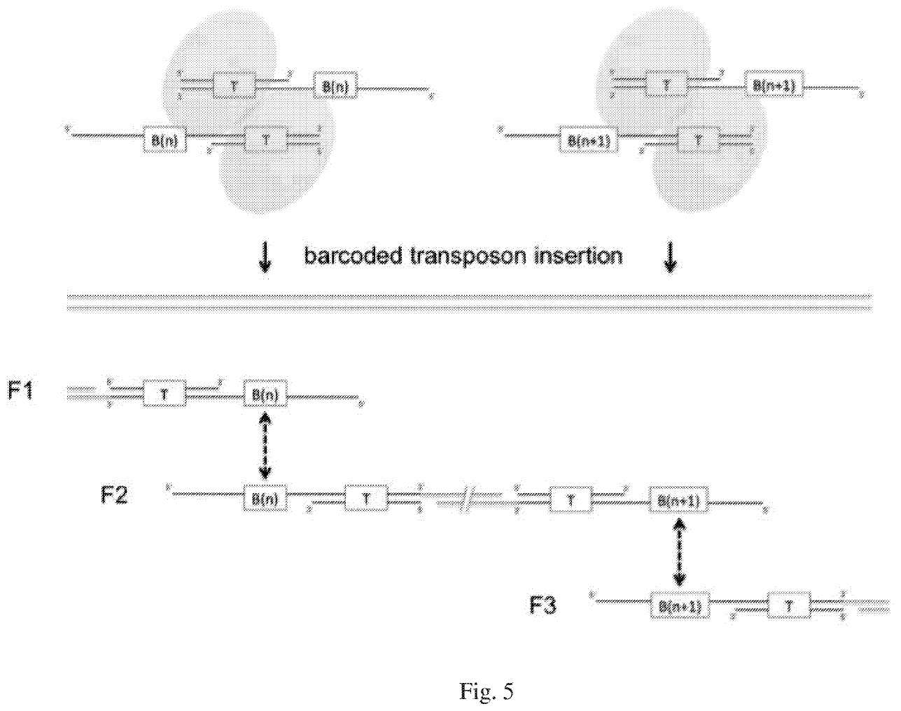

In an embodiment shown in FIG. 3, the transposon DNA is shown as a single stranded overhang. A transposase binds to the double stranded transposase binding site of the transposon DNA and two such complexes dimerize to form the transposome. As shown in FIG. 4, the transposomes randomly capture or otherwise bind to the target single-cell genomic DNA as dimers. Representative transposomes are numbered 1-3. Then, the transposases in the transposome cut the genomic DNA with one transposase cutting an upper strand and one transposase cutting a lower strand to create a genomic DNA fragment. The plurality of transposomes create a plurality of genomic DNA fragments. The transposon DNA is thus inserted randomly into the single-cell genomic DNA, leaving a gap on both ends of the transposition/insertion site. The gap may have any length but a 9 base gap is exemplary. The result is a genomic DNA fragment with a transposon DNA Tnp binding site attached to the 5′ position of an upper strand and a transposon DNA Tnp binding site attached to the 5′ position of a lower strand. Gaps resulting from the attachment or insertion of the transposon DNA are shown. After transposition, the transposase is removed and gap extension is performed to fill the gap and complement the single-stranded overhang originally designed in the transposon DNA as shown in FIG. 5. As a result, primer binding site (“priming site”) sequences are attached to both ends of each genomic DNA fragment as shown in FIG. 5. The primer binding sites are the same for each fragment and are the same for all of the fragments created by the transposomes.

Particular Tn5 transposition systems are described and are available to those of skill in the art. See Goryshin, I. Y. and W. S. Reznikoff, Tn5 in vitro transposition. The Journal of biological chemistry, 1998. 273(13): p. 7367-74; Davies, D. R., et al., Three-dimensional structure of the Tn5 synaptic complex transposition intermediate. Science, 2000. 289(5476): p. 77-85; Goryshin, I. Y., et al., Insertional transposon mutagenesis by electroporation of released Tn5 transposition complexes. Nature biotechnology, 2000. 18(1): p. 97-100 and Steiniger-White, M., I. Rayment, and W. S. Reznikoff, Structure/function insights into Tn5 transposition. Current opinion in structural biology, 2004. 14(1): p. 50-7 each of which are hereby incorporated by reference in their entireties for all purposes. Kits utilizing a Tn5 transposition system for DNA library preparation and other uses are known. See Adey, A., et al., Rapid, low-input, low-bias construction of shotgun fragment libraries by high-density in vitro transposition. Genome biology, 2010. 11(12): p. R119; Marine, R., et al., Evaluation of a transposase protocol for rapid generation of shotgun high-throughput sequencing libraries from nanogram quantities of DNA. Applied and environmental microbiology, 2011. 77(22): p. 8071-9; Parkinson, N.J., et al., Preparation of high-quality next-generation sequencing libraries from picogram quantities of target DNA. Genome research, 2012. 22(1): p. 125-33; Adey, A. and J. Shendure, Ultra-low-input, tagmentation-based whole-genome bisulfite sequencing. Genome research, 2012. 22(6): p. 1139-43; Picelli, S., et al., Full-length RNA-seq from single cells using Smart-seq2. Nature protocols, 2014. 9(1): p. 171-81 and Buenrostro, J. D., et al., Transposition of native chromatin for fast and sensitive epigenomic profiling of open chromatin, DNA-binding proteins and nucleosome position. Nature methods, 2013, each of which is hereby incorporated by reference in its entirety for all purposes. See also WO 98/10077, EP 2527438 and EP 2376517 each of which is hereby incorporated by reference in its entirety. A commercially available transposition kit is marketed under the name NEXTERA and is available from Illumina.

According to one aspect, the method of amplifying DNA further includes genotype analysis of the amplified DNA product. Alternatively, the method of amplifying DNA preferably further includes identifying a polymorphism such as a single nucleotide polymorphism (SNP) in the amplified DNA product. In preferred embodiments, a SNP may be identified in the DNA of an organism by a number of methods well known to those of skill in the art, including but not limited to identifying the SNP by DNA sequencing, by amplifying a PCR product and sequencing the PCR product, by Oligonucleotide Ligation Assay (OLA), by Doublecode OLA, by Single Base Extension Assay, by allele specific primer extension, or by mismatch hybridization. Preferably the identified SNP is associated with a phenotype, including disease phenotypes and desirable phenotypic traits. The amplified DNA generated by using the disclosed method of DNA amplification may also preferably be used to generate a DNA library, including but not limited to genomic DNA libraries, microdissected chromosome DNA libraries, BAC libraries, YAC libraries, PAC libraries, cDNA libraries, phage libraries, and cosmid libraries.

The term “genome” as used herein is defined as the collective gene set carried by an individual, cell, or organelle. The term “genomic DNA” as used herein is defined as DNA material comprising the partial or full collective gene set carried by an individual, cell, or organelle.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. The term also refers to both double- and single-stranded molecules. Unless otherwise specified or required, any embodiment of this invention that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule. This alphabetical representation can be input into databases in a computer having a central processing unit and used for bioinformatics applications such as functional genomics and homology searching.

The terms “DNA,” “DNA molecule” and “deoxyribonucleic acid molecule” refer to a polymer of deoxyribonucleotides. DNA can be synthesized naturally (e.g., by DNA replication). RNA can be post-transcriptionally modified. DNA can also be chemically synthesized. DNA can be single-stranded (i.e., ssDNA) or multi-stranded (e.g., double stranded, i.e., dsDNA).

The terms “nucleotide analog,” “altered nucleotide” and “modified nucleotide” refer to a non-standard nucleotide, including non-naturally occurring ribonucleotides or deoxyribonucleotides. In certain exemplary embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; 0- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NEM, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the terms “complementary” and “complementarity” are used in reference to nucleotide sequences related by the base-pairing rules. For example, the sequence 5′-AGT-3′ is complementary to the sequence 5′-ACT-3′. Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. The degree of complementarity between nucleic acid strands has significant effects on the efficiency and strength of hybridization between nucleic acid strands.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the melting temperature of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See, e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted.

“Low stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 g/ml denatured salmon sperm DNA followed by washing in a solution comprising 5×SSPE, 0.1% SDS at 42° C. when a probe of about 500 nucleotides in length is employed.

“Medium stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5×Denhardt's reagent and 100 g/ml denatured salmon sperm DNA followed by washing in a solution comprising 1.0×SSPE, 1.0% SDS at 42° C. when a probe of about 500 nucleotides in length is employed.

“High stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5×Denhardt's reagent and 100 g/ml denatured salmon sperm DNA followed by washing in a solution comprising 0.1×SSPE, 1.0% SDS at 42° C. when a probe of about 500 nucleotides in length is employed.

In certain exemplary embodiments, cells are identified and then a single cell or a plurality of cells are isolated. Cells within the scope of the present disclosure include any type of cell where understanding the DNA content is considered by those of skill in the art to be useful. A cell according to the present disclosure includes a cancer cell of any type, hepatocyte, oocyte, embryo, stem cell, iPS cell, ES cell, neuron, erythrocyte, melanocyte, astrocyte, germ cell, oligodendrocyte, kidney cell and the like. According to one aspect, the methods of the present invention are practiced with the cellular DNA from a single cell. A plurality of cells includes from about 2 to about 1,000,000 cells, about 2 to about 10 cells, about 2 to about 100 cells, about 2 to about 1,000 cells, about 2 to about 10,000 cells, about 2 to about 100,000 cells, about 2 to about 10 cells or about 2 to about 5 cells.

Nucleic acids processed by methods described herein may be DNA and they may be obtained from any useful source, such as, for example, a human sample. In specific embodiments, a double stranded DNA molecule is further defined as comprising a genome, such as, for example, one obtained from a sample from a human. The sample may be any sample from a human, such as blood, serum, plasma, cerebrospinal fluid, cheek scrapings, nipple aspirate, biopsy, semen (which may be referred to as ejaculate), urine, feces, hair follicle, saliva, sweat, immunoprecipitated or physically isolated chromatin, and so forth. In specific embodiments, the sample comprises a single cell. In specific embodiments, the sample includes only a single cell.

In particular embodiments, the amplified nucleic acid molecule from the sample provides diagnostic or prognostic information. For example, the prepared nucleic acid molecule from the sample may provide genomic copy number and/or sequence information, allelic variation information, cancer diagnosis, prenatal diagnosis, paternity information, disease diagnosis, detection, monitoring, and/or treatment information, sequence information, and so forth.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, such that each single cell is placed in a single well.

Methods for manipulating single cells are known in the art and include fluorescence activated cell sorting (FACS), flow cytometry (Herzenberg., PNAS USA 76:1453-55 1979), micromanipulation and the use of semi-automated cell pickers (e.g. the Quixell™ cell transfer system from Stoelting Co.). Individual cells can, for example, be individually selected based on features detectable by microscopic observation, such as location, morphology, or reporter gene expression. Additionally, a combination of gradient centrifugation and flow cytometry can also be used to increase isolation or sorting efficiency.

Once a desired cell has been identified, the cell is lysed to release cellular contents including DNA, using methods known to those of skill in the art. The cellular contents are contained within a vessel or a collection volume. In some aspects of the invention, cellular contents, such as genomic DNA, can be released from the cells by lysing the cells. Lysis can be achieved by, for example, heating the cells, or by the use of detergents or other chemical methods, or by a combination of these. However, any suitable lysis method known in the art can be used. For example, heating the cells at 72° C. for 2 minutes in the presence of Tween-20 is sufficient to lyse the cells. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5): e42 (2006)); or lysis can be achieved with a protease such as Proteinase K or by the use of chaotropic salts such as guanidine isothiocyanate (U.S. Publication No. 2007/0281313). Amplification of genomic DNA according to methods described herein can be performed directly on cell lysates, such that a reaction mix can be added to the cell lysates. Alternatively, the cell lysate can be separated into two or more volumes such as into two or more containers, tubes or regions using methods known to those of skill in the art with a portion of the cell lysate contained in each volume container, tube or region. Genomic DNA contained in each container, tube or region may then be amplified by methods described herein or methods known to those of skill in the art.

A nucleic acid used in the invention can also include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

The expression “amplification” or “amplifying” refers to a process by which extra or multiple copies of a particular polynucleotide are formed.

The DNA amplified according to the methods described herein may be sequenced and analyzed using methods known to those of skill in the art. Determination of the sequence of a nucleic acid sequence of interest can be performed using a variety of sequencing methods known in the art including, but not limited to, sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

The amplified DNA can be sequenced by any suitable method. In particular, the amplified DNA can be sequenced using a high-throughput screening method, such as Applied Biosystems' SOLiD sequencing technology, or Illumina's Genome Analyzer. In one aspect of the invention, the amplified DNA can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

“Shotgun sequencing” refers to a method used to sequence very large amount of DNA (such as the entire genome). In this method, the DNA to be sequenced is first shredded into smaller fragments which can be sequenced individually. The sequences of these fragments are then reassembled into their original order based on their overlapping sequences, thus yielding a complete sequence. “Shredding” of the DNA can be done using a number of difference techniques including restriction enzyme digestion or mechanical shearing. Overlapping sequences are typically aligned by a computer suitably programmed. Methods and programs for shotgun sequencing a cDNA library are well known in the art.

The amplification and sequencing methods are useful in the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the genomic DNA in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

As used herein, the term “biological sample” is intended to include, but is not limited to, tissues, cells, biological fluids and isolates thereof, isolated from a subject, as well as tissues, cells and fluids present within a subject.

In certain exemplary embodiments, electronic apparatus readable media comprising one or more genomic DNA sequences described herein is provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon one or more expression profiles described herein.

As used herein, the term “electronic apparatus” is intended to include any suitable computing or processing apparatus or other device configured or adapted for storing data or information. Examples of electronic apparatuses suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising one or more expression profiles described herein.

A variety of software programs and formats can be used to store the genomic DNA information of the present invention on the electronic apparatus readable medium. For example, the nucleic acid sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon one or more expression profiles described herein.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### Example I

### General Protocol

The following general protocol is useful for whole genome amplification. A single cell is lysed in lysis buffer. Transposome is formed by incubating equal molar of transposon DNA and Tn5 transposase at room temperature for 1 hour. The transposome and transposition buffer are added to the cell lysis which is mixed well and is incubated at 55° C. for 10 minutes. 1 mg/ml protease is added after the tranposition to remove the transpoase from binding to the single cell genomic DNA. Deep vent (exo-) DNA Polymerase (New England Biolabs), dNTP, PCR reaction buffer and primers are added to the reaction mixture which is heated to 72° C. for 10 min to fill in the gap generated from the transposon insertion. The reaction mixture is loaded to the microfluidic device to form micro droplets. The droplets containing single cell genomic DNA template, DNA polymerase, dNTP, reaction buffer and primer are collected into PCR tubes. 40 to 60 cycles of PCR reaction are performed to amplify the single cell genomic DNA. The number of cycles is selected to drive the amplification reaction in the droplets to saturation. The droplets are lysed and the amplification products are purified for further analysis like high through put deep sequencing.

### Example II

### Combining Transposase with Transposon DNA

Tn5 transposase (Epicentre) is mixed with transposon DNA in equal molar number in a buffer containing EDTA and incubated at room temperature for 10-60 minutes. The final transposome concentration is 0.1-10 μM. The transposon DNA construct has a double stranded 19 bp transposase binding site on one end, and a priming site on the other end. The single stranded priming site forms a 5′ protruding end. Barcode sequences with variable length and sequence complexity could be designed as needed between the 19 bp binding site and the priming site. The transposome may be diluted by many folds in 50% Tris-EDTA and 50% glycerol solution and preserved at −20° C.

### Example III

### Cell Lysis

A cell is selected, cut from a culture dish, and dispensed in a tube using a laser dissection microscope (LMD-6500, Leica) as follows. The cells are plated onto a membrane-coated culture dish and observed using bright field microscopy with a 10× objective (Leica). A UV laser is then used to cut the membrane around an individually selected cell such that it falls into the cap of a PCR tube. The tube is briefly centrifuged to bring the cell down to the bottom of the tube. 3-5 μl lysis buffer (30 mM Tris-Cl PH 7.8, 2 mM EDTA, 20 mM KCl, 0.2% Triton X-100, 500 μg/ml Qiagen Protease) is added to the side of the PCR tube and span down. The captured cell is then thermally lysed using the using following temperature schedule on PCR machine: 50° C. 3 hours, 75° C. 30 minutes. Alternatively, mouth pipette a single cell into a low salt lysis buffer containing EDTA and protease such as QIAGEN protease (QIAGEN) at a concentration of 10-5000 μg/mL. The incubation condition varies based on the protease that is used. In the case of QIAGEN protease, the incubation would be 37-55° C. for 1-4 hrs. The protease is then heat inactivated up to 80° C. and further inactivated by specific protease inhibitors such as 4-(2-Aminoethyl) benzenesulfonyl fluoride hydrochloride (AEBSF) or phenylmethanesulfonyl fluoride (PMSF) (Sigma Aldrich). The cell lysis is preserved at −80° C.

Alternatively, human BJ cell lines cultured in a Petri dish are trypsinized and collected into an Eppendordf low binding tube. The cells are washed with PBS to remove cell growth medium and resuspended into 150 mM NaCl buffer. The cells are further diluted to −5 cells/ul and plated onto a membrane coated culture dish. Single cells are picked into 5 ul of cell lysis buffer (20 mM Tris pH 8.0, 20 mM NaCl, 0.2% Triton X-100, 15 mM DTT, 1 mM EDTA, lmg/ml Qiagen protease) by a mouth pipetting system. The captured cell is then thermally lysed using following temperature schedule on PCR machine: 50° C. 3 hours, 70° C. 30 minutes. The lysed cells are stored at −80 C before digital amplification via transposon insertion (DIANTI).

### Example IV

### Transposition

The single cell lysis and the transposome are mixed in a buffer system containing 1-100 mM Mg²⁺ and optionally 1-100 mM Mn²⁺ or Co²⁺ or Ca²⁺ as well and incubate at 37-55° C. for 5-240 minutes. The reaction volume varies depending on the cell lysis volume. The amount of transposome added in the reaction could be readily tuned depending on the desired fragmentation size. The transposition reaction is stopped by chelating Mg²⁺ using EDTA and optionally EGTA or other chelating agents for ions. Optionally, short double stranded DNA could be added to the mixture as a spike-in. The residue transposome is inactivated by protease digestion such as QIAGEN protease at a final concentration 1-500 μg/mL at 37-55° C. for 10-60 minutes. The protease is then inactivated by heat and/or protease inhibitor, such as AEBSF.

### Example V

### Gap Filling

After transposition and transposase removal, a PCR reaction mixture including Mg²⁺, dNTP mix, primers and a thermal stable DNA polymerase such as Deep Vent (exo-)DNA Polymerase (New England Biolabs) is added to the solution at a suitable temperature and for a suitable time period to fill the 9 bp gap left by the transposition reaction. The gap filling incubation temperature and time depends on the specific DNA polymerase used. After the reaction, the DNA polymerase is optionally inactivated by heating and/or protease treatment such as QIAGEN protease. The protease, if used, is then inactivated by heat and/or protease inhibitor.

### Example VI

### Generation of Microdroplets and Isolation of Each DNA Fragment in a Separate Microdroplet and Amplification

According to one aspect, general methods known to those of skill in the art are used to create droplets of PCR amplification reaction reagents where reactions are carried out in each droplet to amplify a DNA fragment within the droplet. The gap filled double stranded products from the above example including the DNA fragments with primer binding sites are added to PCR reaction reagents in an aqueous medium which is then combined with oil and the combination results in droplets where the number of droplets exceeds the number of gap filled double stranded products such that a single gap filled double stranded product is isolated within a single droplet along with sufficient PCR reaction reagents. The droplets are then subject to PCR conditions to PCR amplify each DNA fragment within each droplet. Suitable emulsion droplet amplification methods are known to those of skill in the art and include those described in Mazutis, L., et al. Single-cell analysis and sorting using droplet-based microfluidics, Nature Protocols, 2013, 8, p. 870-891; Williams, R, et al. Amplification of complex gene libraries by emulsion PCR, Nature Methods, 2006, 3, p. 545-550; Fu, Y, et al. Uniform and accurate single-cell sequencing based on emulsion whole-genome amplification. Proceedings of the National Academy of Sciences of the United States of America, 2015, 112(38): p. 11923-8; Sidore, A. M., et al. Enhanced sequencing coverage with digital droplet multiple displacement amplification. Nucleic Acids Research. 2015, December 23; Nishikawa, Y, et al. Monodisperse picoliter droplets for low-bias and contamination-free reactions in single-cell whole genome amplification. PLoS One. 2015, Sep. 21; Rhee, M., et al. Digital droplet multiple displacement amplification (ddMDA) for whole genome sequencing of limited DNA samples. PLoS One. 2016. May 4; Guo, M. T., et al. Droplet microfluidics for high-throughput biological assays, Lab on a Chip, 2012, 12, p. 2146-2155; Chabert, M., et al. Automated microdroplet platform for sample manipulation and polymerase chain reaction, Analytical Chemistry, 2006, 78(22), p. 7722-′7′728; Kiss, M. M., High-throughput quantitative polymerase chain reaction in picoliter droplets, Analytical Chemistry, 2008, 80(23), p. 8975-8981; Lan, F., et al. Droplet barcoding for massively parallel single-molecule deep sequencing, Nature Communications, 2016, 7(11784) each of which is hereby incorporated by reference in its entirety. Suitable oil phases are known to those of skill in the art in which an aqueous phase spontaneously results in aqueous droplets or isolated volumes or compartments surrounded by the oil phase. Exemplary oils include the QX200™ Droplet Generation Oil for Evagreen (Bio-Rad), 008-FluoroSurfactant in FIFE 7500 (RAN Biotechnologies), Pico-Surf™ 1 (Dolomite Microfluidics), Proprietary Oil Surfactants (RainDance Technologies), fluorosurfactants and fluorinated oils discussed in Mazutis, L., et al. Single-cell analysis and sorting using droplet-based microfluidics, Nature Protocols, 2013, 8, p. 870-891, and other surfactants and oils described in Baret, J.-C. Lab on a Chip, 2012, 12, p. 422-433 each of which is hereby incorporated by reference in its entirety.

Useful microfluidic devices for carrying out single cell whole genome amplification are described in Wang et al., Cell 150(2):402-412 (2012), de Bourcy CFA, PLOS ONE 9(8):e105585 (2014), Gole et al., Nat Biotechnol 31(12):1126-1132 (2013) and Yu et al., Anal Chem 86(19):9386-9390 (2014); Fu, Y, et al. Uniform and accurate single-cell sequencing based on emulsion whole-genome amplification. Proceedings of the National Academy of Sciences of the United States of America, 2015, 112(38): p. 11923-8; Sidore, A. M., et al. Enhanced sequencing coverage with digital droplet multiple displacement amplification. Nucleic Acids Research. 2015, Dec. 23; Nishikawa, Y, et al. Monodisperse picoliter droplets for low-bias and contamination-free reactions in single-cell whole genome amplification. PLoS One. 2015, Sep. 21; Rhee, M., et al. Digital droplet multiple displacement amplification (ddMDA) for whole genome sequencing of limited DNA samples. PLoS One. 2016. May 4; Lan, F., et al. Droplet barcoding for massively parallel single-molecule deep sequencing, Nature Communications, 2016, 7(11784) each of which is hereby incorporated by reference in its entirety. Such devices allow for avoidance of contaminations and high throughput analyses of multiple single molecules or single cells in parallel. The small total reaction volumes (microliters to nanoliters or picoliters) of the microfluidic devices not only facilitate the efficiency of reactions but also allow significant cost reduction for enzymes and reagents used.

### Example VII

### Amplification of a DNA Fragment Isolated in a Microdroplet

The gap filled DNA fragments from the above example were loaded into a microfluidic chip to generate between 1 and 100 million micro droplets. The microfluidic chip design was modified from a conventional flow-focusing droplet generation design as provided by Macosko et al. Cell 161 (5), 2015 hereby incorporated by reference in its entirety. The microfluidic chip design included a hydrophobic liquid inlet (referred to as an oil inlet), a DNA solution or aqueous phase inlet, a combination zone for combining the oil phase and the aqueous phase connected in fluid communication by microchannels further connected in fluid communication to an emulsion droplet outlet region. Surface area and sharp angles along the aqueous flow path were minimized compared to the design of Macosko et al. Cell 161 (5), 2015 to prevent sticking of DNA fragments on surfaces of the microfluidic chip design. The oil phase inlet included a filter commonly used in microfluidic designs, such as filtering squares. The aqueous phase inlet also included a filter commonly used in microfluidic designs, such as filtering squares, however the surface area of the filtering squares was reduced to minimize surface area contacted by the aqueous phase. A suitable hydrophobic phase is one that generates aqueous droplets when an aqueous media is introduced into the hydrophobic phase. An exemplary hydrophobic phase includes a hydrophobic liquid, such as an oil, such as a fluorinated oil, such as 3-ethoxyperfluoro(2-methylhexane), and a surfactant. Surfactants are well known to those of skill in the art. An exemplary hydrophobic phase including a suitable oil and a surfactant is commercially available as QX200™ Droplet Generation Oil for Evagreen (Bio-Rad), a hydrophobic surfactant-containing liquid that does not mix with aqueous solution or adversely affect biochemical reactions in aqueous solution. Other suitable oil and surfactant combinations are commercially available or known to those of skill in the art. When the oil phase and the aqueous phase are combined in the combination region or the emulsion droplet outlet region, the aqueous phase will spontaneously form droplets surrounded by the oil phase. According to one aspect, a flush volume of a hydrophobic fluid, such as an oil which may not contain a surfactant as none is needed for a flush volume, upstream of the aqueous phase either within the microfluidic design or within the syringe or injector used to input the aqueous phase into the microfluidic design is used to displace any aqueous phase that may otherwise occupy a dead volume to minimize loss of original aqueous phase introduced into the microfluidic chip design. Useful microfluidic chip designs can be created using AutoCAD software (Autodesk Inc.) and can be printed by CAD Art Services Inc. into a photomask for microfluidic fabrication. Molds or masters can be created using conventional techniques as described in Mazutis et al. Nature Protocols 8 (5), 2013 hereby incorporated by reference in its entirety. Microfluidic chips can be made from the master by curing uncured polydimethyl siloxane (PDMS)(Dow Corning Sylgard 184) poured onto the master and heated to curing to create a surface with trenches or circuits. Inlet and outlet holes are created and the cured surface with the circuits is placed against a glass slide and secured to create the microchannels and the microfluidic chip. Before use, the interior of the microfluidic chip can be treated with a compound for improving the hydrophobicity of the interior of the microfluidic chip and washed to remove potential contamination.

For the experiments conducted herein, each microfluidic chip was treated with Aquapel (Aquapel) to make the channel surfaces hydrophobic. Before starting each experiment, the device was washed with nuclease-free water to remove potential contamination, and then washed with droplet generation oil such as the QX200™ Bio-Rad Droplet Generation Oil for Evagreen. The droplet generation oil is in a syringe connected to the oil inlet of the microfluidic chip via polyethylene tubing (Scientific Commodities #BB31695-PE/2). The outlet of the chip is connected to a 2 ml DNA LoBind tube via polyethylene tubing for droplet collection.

To load genomic DNA (gDNA) solution into the microfluidic chip without dead volume, a 1 ml syringe connected to a 140 cm-long polyethylene tubing via syringe needle was pre-filled with 3-ethoxyperfluoro(2-methylhexane) (“HFE oil”). The gDNA solution (prepared from transposon insertion and gap filling) was then sucked into the tubing without touching the syringe needle or syringe, where dead volume occurs. To distinguish HFE oil from gDNA solution inside the polyethylene tube (which are both transparent), a small amount of air was sucked into the polyethylene tube before sucking in the gDNA solution to separate both types of liquids. This method ensures that all gDNA solution be pumped into the chip without remaining in the syringe needle or the syringe; the solution is pushed fully into the chip by the HFE oil that does not mix with it.

When the gDNA solution and the droplet generation oil were combined in the microfluidic device, droplets formed spontaneously in the flow circuit. The droplets were then aliquoted into PCR tubes for thermocycling for amplification. A PCR reaction was performed in a thermal cycler according to the following schedule:

| Cycle step | Temperature | Time | Cycles |
|---|---|---|---|
| Initial Denaturation | 95° C. | 5 minutes | 1 |
| Denaturation | 95° C. | 2 minutes | 40~60 |
| Annealing | 64° C. | 2 minutes | |
| Extension | 72° C. | 10 minutes | |
| Final Extension | 72° C. | 20 minutes | 1 |
| Hold | 4° C. | ∞ | |

Afterwards, 75 μl of perfluorooctanol (TCI Chemicals) was added to each PCR tube; after shaking by hand and centrifugation, all droplets were lysed and aqueous solution containing DNA amplification products were collected into an Eppendorf low binding tube and purified by Zymo Research DNA Clean & Concentrator-5 and pooled together for downstream analyses. The concentration of the purified DNA products is measured by Qubit 2.0 fluorometer. 10 ng amplified DNA is used for one qPCR primer locus to determine the amplification yield and evenness of the amplification resulting from the transposition system and emulsion droplet amplification method.

1 ug amplified DNA product is used as input to make an Illumine sequencing library with Illumina TruSeq DNA PCR-free library preparation kits. The input DNA are first sonicated on a Covaris sonicator and a size selection is performed to enrich DNA fragments with length around 300 bp. Three samples from human cells, SC2, SC3d2 and SC6 are loaded to three lanes of an Illumina HiSeq 4000 sequencing system. Around 60G raw data are acquired per sample.

The sequencing data are aligned to a human reference genome by Burrows-Wheeler Aligner (BWA). The coverage is determined by plotting the Lorenz curve of the mapped sequencing reads. The SNVs are determined by SAMtools. The allele drop out (ADO) rate is calculated by ratio of the undetected and the actual heterozygous SNVs in a single cell.

### Example VIII

### DNA Fragment Size Analysis

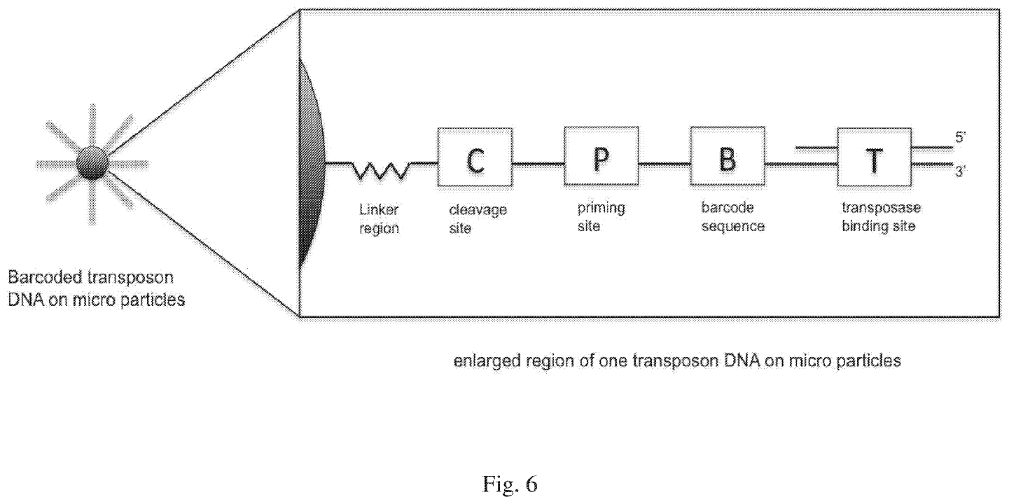

According to one aspect, the Tn5 transposome preparation and the transposition reaction conditions can be varied to result in different DNA fragment sizes. The Tn5 transposition efficiency and the insertion density could be tuned at will within a large range. After single cell genomic DNA amplification as described herein, more than 1 microgram of amplification product is generated from the amplification and the product size distribution was probed by a DNA BioAnalyzer, the results of which are shown in FIG. 6. The x-axis is the fragment size, and the y-axis is the relative amount reflected by the fluorescence intensity with an arbitrary unit. The two sharp peaks at both sides of the image are the two spike-in DNA fragments of 35 bp and 10380 bp, respectively. The average length of the amplification product was above 3000 bp in size.

The qPCR results with 8 different loci across the whole genome of human cells showed very uniform amplification as indicated in Table 1 below.

| Human | | | |
|---|---|---|---|
| genome loci | SC2 | SC3d2 | SC6 |
| L1 | 24.9 | 22.1 | 24.1 |
| L2 | 24.2 | 23.7 | 24.4 |
| L3 | 24.1 | 26.1 | 24.1 |
| L4 | 28.6 | 24.4 | 23.6 |
| L5 | 24.0 | 24.3 | 24.8 |
| L6 | 26.2 | 24.4 | 25.9 |
| L7 | 26.7 | 24.1 | 28.1 |
| L8 | 23.7 | 25.5 | 23.5 |

To further investigate the amplification efficiency, libraries from the amplification products of all three single cells were created and sequenced to 30× on an Illumina high-throughput sequencing system. The sequencing data were mapped to a reference human genome with Burrows-Wheeler Aligner (BWA). An average coverage of 90% of reference human genome and an average allele drop rate (ADO) of 30% were achieved after analysis as indicated in Table 2 below, which surpassed the currently available commercialized single cell whole genome amplification kits (Table. 2).

| Kit | WGA method | Coverage | ADO |
|---|---|---|---|
| N/A | DIANTI | 90% | 30% |
| Sigma- | DOP-PCR | 39% | 76% |
| Aldrich | | | |
| GE | MDA | 82% | 35% |
| Yikon | MALBAC | 72% | 45% |

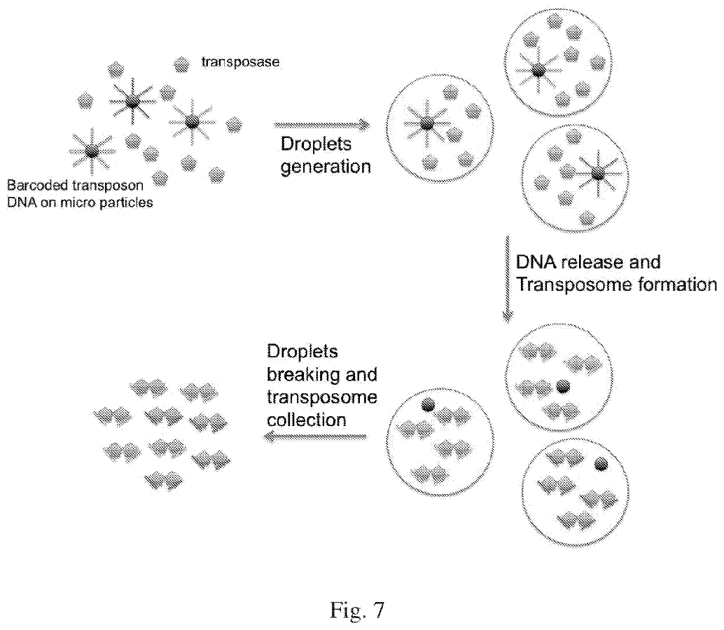

A read depth analysis as shown in FIG. 7 of three single human cells where whole genomes were amplified using the transposition system and droplet emulsion amplification technique described herein showed very uniform amplification efficiency across the whole human genome, which is useful in improving the resolution and accuracy of copy number variations (CNV) calling.

### Example IX

### Separation Techniques

Following amplification, it may be desirable to separate the amplification products of several different lengths from each other, from the template, and from excess primers for the purpose of analysis.

In one embodiment, amplification products are separated by agarose, agarose-acrylamide or polyacrylamide gel electrophoresis using standard methods (Sambrook et al., “Molecular Cloning,” A Laboratory Manual, 2d Ed., Cold Spring Harbor Laboratory Press, New York, 13.7-13.9:1989). Gel electrophoresis techniques are well known in the art.

Alternatively, chromatographic techniques may be employed to effect separation. There are many kinds of chromatography which may be used in the present disclosure: adsorption, partition, ion-exchange, and molecular sieve, as well as many specialized techniques for using them including column, paper, thin-layer and gas chromatography (Freifelder, Physical Biochemistry Applications to Biochemistry and Molecular Biology, 2nd ed. Wm. Freeman and Co., New York, N.Y., 1982). Yet another alternative is to capture nucleic acid products labeled with, for example, biotin or antigen with beads bearing avidin or antibody, respectively.

Microfluidic techniques include separation on a platform such as microcapillaries, including by way of example those designed by ACLARA BioSciences Inc., or the LabChip™ by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR™ apparatus for collection and amplification of nucleic acids from a specimen. U.S. Pat. Nos. 5,304,487, 5,296,375, and 5,856,174 describe apparatus and methods incorporating the various processing and analytical operations involved in nucleic acid analysis and are incorporated herein by reference.

In some embodiments, it may be desirable to provide an additional, or alternative means for analyzing the amplified DNA. In these embodiments, microcapillary arrays are contemplated to be used for the analysis. Microcapillary array electrophoresis generally involves the use of a thin capillary or channel that may or may not be filled with a particular separation medium. Electrophoresis of a sample through the capillary provides a size based separation profile for the sample. Microcapillary array electrophoresis generally provides a rapid method for size-based sequencing, PCR product analysis, and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, for example, Jacobson et al., Anal Chem, 66:1107-1113, 1994; Effenhauser et al., Anal Chem, 66:2949-2953, 1994; Harrison et al., Science, 261:895-897, 1993; Effenhauser et al., Anal Chem, 65:2637-2642, 1993; Manz et al., J. Chromatogr 593:253-258, 1992; and U.S. Pat. No. 5,904,824, incorporated herein by reference. Typically, these methods comprise photolithographic etching of micron scale channels on a silica, silicon, or other crystalline substrate or chip, and can be readily adapted for use in the present disclosure.

Tsuda et al. (Anal Chem, 62:2149-2152, 1990) describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined, or molded into planar substrates, are filled with an appropriate separation/sieving matrix. Typically, a variety of sieving matrices known in the art may be used in the microcapillary arrays. Examples of such matrices include, e.g., hydroxyethyl cellulose, polyacrylamide, agarose, and the like. Generally, the specific gel matrix, running buffers, and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea to denature nucleic acids in the sample.

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods in the art are summarized in Methods in Enzymology, Vol. 193: “Mass Spectrometry” (J. A. McCloskey, editor), 1990, Academic Press, New York.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Reviews summarizing this field include (Schram, Methods Biochem Anal, 34:203-287, 1990) and (Crain, Mass Spectrometry Reviews, 9:505-554, 1990), here incorporated herein by reference. The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Koster et al., Biomedical Environmental Mass Spectrometry 14:111-116, 1987).

Two ionization/desorption techniques are electrospray/ionspray (ES) and matrix-assisted laser desorption/ionization (MALDI). ES mass spectrometry was introduced by Fenn et al., J. Phys. Chem. 88; 4451-59, 1984; PCT Application No. WO 90/14148 and its applications are summarized in review articles, for example, Smith et al., Anal Chem 62:882-89, 1990, and Ardrey, Electrospray Mass Spectrometry, Spectroscopy Europe, 4:10-18, 1992. As a mass analyzer, a quadrupole is most frequently used. The determination of molecular weights in femtomole amounts of sample is very accurate due to the presence of multiple ion peaks that can be used for the mass calculation.

MALDI mass spectrometry, in contrast, can be particularly attractive when a time-of-flight (TOF) configuration is used as a mass analyzer. The MALDI-TOF mass spectrometry was introduced by (Hillenkamp et al., Biological Mass Spectrometry eds. Burlingame and McCloskey, Elsevier Science Publishers, Amsterdam, pp. 49-60, 1990). Since, in most cases, no multiple molecular ion peaks are produced with this technique, the mass spectra, in principle, look simpler compared to ES mass spectrometry. DNA molecules up to a molecular weight of 410,000 daltons could be desorbed and volatilized (Williams et al., Science, 246:1585-87, 1989). More recently, the use of infrared lasers (IR) in this technique (as opposed to UV-lasers) has been shown to provide mass spectra of larger nucleic acids such as synthetic DNA, restriction enzyme fragments of plasmid DNA, and RNA transcripts up to a size of 2180 nucleotides (Berkenkamp et al., Science, 281:260-2, 1998). Berkenkamp also describes how DNA and RNA samples can be analyzed by limited sample purification using MALDI-TOF IR.

In Japanese Patent No. 59-131909, an instrument is described that detects nucleic acid fragments separated either by electrophoresis, liquid chromatography or high speed gel filtration. Mass spectrometric detection is achieved by incorporating into the nucleic acids atoms that normally do not occur in DNA such as S, Br, I or Ag, Au, Pt, Os, Hg.

Labeling hybridization oligonucleotide probes with fluorescent labels is a well known technique in the art and is a sensitive, nonradioactive method for facilitating detection of probe hybridization. More recently developed detection methods employ the process of fluorescence energy transfer (FET) rather than direct detection of fluorescence intensity for detection of probe hybridization. FET occurs between a donor fluorophore and an acceptor dye (which may or may not be a fluorophore) when the absorption spectrum of one (the acceptor) overlaps the emission spectrum of the other (the donor) and the two dyes are in close proximity. Dyes with these properties are referred to as donor/acceptor dye pairs or energy transfer dye pairs. The excited-state energy of the donor fluorophore is transferred by a resonance dipole-induced dipole interaction to the neighboring acceptor. This results in quenching of donor fluorescence. In some cases, if the acceptor is also a fluorophore, the intensity of its fluorescence may be enhanced. The efficiency of energy transfer is highly dependent on the distance between the donor and acceptor, and equations predicting these relationships have been developed by Forster, Ann Phys 2:55-75, 1948. The distance between donor and acceptor dyes at which energy transfer efficiency is 50% is referred to as the Forster distance (Ro). Other mechanisms of fluorescence quenching are also known in the art including, for example, charge transfer and collisional quenching.

Energy transfer and other mechanisms that rely on the interaction of two dyes in close proximity to produce quenching are an attractive means for detecting or identifying nucleotide sequences, as such assays may be conducted in homogeneous formats. Homogeneous assay formats differ from conventional probe hybridization assays that rely on the detection of the fluorescence of a single fluorophore label because heterogeneous assays generally require additional steps to separate hybridized label from free label. Several formats for FET hybridization assays are reviewed in Nonisotopic DNA Probe Techniques (Academic Press, Inc., pgs. 311-352, 1992).

Homogeneous methods employing energy transfer or other mechanisms of fluorescence quenching for detection of nucleic acid amplification have also been described. Higuchi et al. (Biotechnology 10:413-417, 1992), discloses methods for detecting DNA amplification in real-time by monitoring increased fluorescence of ethidium bromide as it binds to double-stranded DNA. The sensitivity of this method is limited because binding of the ethidium bromide is not target specific and background amplification products are also detected. Lee et al. (Nucleic Acids Res 21:3761-3766, 1993), discloses areal-time detection method in which a doubly-labeled detector probe is cleaved in a target amplification-specific manner during PCR™. The detector probe is hybridized downstream of the amplification primer so that the 5′-3′ exonuclease activity of Taq polymerase digests the detector probe, separating two fluorescent dyes, which then form an energy transfer pair. Fluorescence intensity increases as the probe is cleaved. Published PCT application WO 96/21144 discloses continuous fluorometric assays in which enzyme-mediated cleavage of nucleic acids results in increased fluorescence. Fluorescence energy transfer is suggested for use, but only in the context of a method employing a single fluorescent label that is quenched by hybridization to the target.

Signal primers or detector probes that hybridize to the target sequence downstream of the hybridization site of the amplification primers have been described for use in detection of nucleic acid amplification (U.S. Pat. No. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product that may be detected as an indication of target amplification. The secondary amplification products generated from signal primers may be detected by means of a variety of labels and reporter groups, restriction sites in the signal primer that are cleaved to produce fragments of a characteristic size, capture groups, and structural features such as triple helices and recognition sites for double-stranded DNA binding proteins.

Many donor/acceptor dye pairs are known in the art and may be used in the present disclosure. These include but are not limited to: fluorescein isothiocyanate (FITC)/tetramethylrhodamine isothiocyanate (TALIC), FITC/Texas Red™ Molecular Probes, FITC/N-hydroxysuccmimidyl 1-pyrenebutyrate (PYB), FITC/eosin isothiocyanate (EITC), N-hydroxysuccinimidyl 1-pyrenesulfonate (PYS)/FITC, FITC/Rhodamine X, FITC/tetramethylrhodamine (TAIVIRA), and others. The selection of a particular donor/acceptor fluorophore pair is not critical. For energy transfer quenching mechanisms it is only necessary that the emission wavelengths of the donor fluorophore overlap the excitation wavelengths of the acceptor, i.e., there must be sufficient spectral overlap between the two dyes to allow efficient energy transfer, charge transfer, or fluorescence quenching. P-(dimethyl aminophenylazo) benzoic acid (DABCYL) is a non-fluorescent acceptor dye which effectively quenches fluorescence from an adjacent fluorophore, e.g., fluorescein or 5-(2′-aminoethyl) aminonaphthalene (EDANS). Any dye pairs that produce fluorescence quenching in the detector nucleic acids are suitable for use in the methods of the disclosure, regardless of the mechanism by which quenching occurs. Terminal and internal labeling methods are both known in the art and may be routinely used to link the donor and acceptor dyes at their respective sites in the detector nucleic acid.

Specifically contemplated in the present disclosure is the use or analysis of amplified products by microarrays and/or chip-based DNA technologies such as those described by (Hacia et al., Nature Genet, 14:441-449, 1996) and (Shoemaker et al., Nature Genetics, 14:450-456, 1996). These techniques involve quantitative methods for analyzing large numbers of genes rapidly and accurately. By tagging genes with oligonucleotides or using fixed probe arrays, chip technology can be employed to segregate target molecules as high density arrays and screen these molecules on the basis of hybridization (Pease et al., Proc Natl Acad Sci USA, 91:5022-5026, 1994; Fodor et al, Nature, 364:555-556, 1993).

Also contemplated is the use of BioStar's OIA technology to quantitate amplified products. OIA uses the mirror-like surface of a silicon wafer as a substrate. A thin film optical coating and capture antibody is attached to the silicon wafer. White light reflected through the coating appears as a golden background color. This color does not change until the thickness of the optical molecular thin film is changed.

When a positive sample is applied to the wafer, binding occurs between the ligand and the antibody. When substrate is added to complete the mass enhancement, a corresponding change in color from gold to purple/blue results from the increased thickness in the molecular thin film. The technique is described in U.S. Pat. No. 5,541,057, herein incorporated by reference.

Amplified DNA may be quantitated using the Real-Time PCR technique (Higuchi et al., Biotechnology 10:413-417, 1992). By determining the concentration of the amplified products that have completed the same number of cycles and are in their linear ranges, it is possible to determine the relative concentrations of the specific target sequence in the original DNA mixture. The goal of a Real-Time PCR experiment is to determine the abundance of a particular RNA or DNA species relative to the average abundance of all RNA or DNA species in the sample.

The Luminex technology allows the quantitation of nucleic acid products immobilized on color coded microspheres. The magnitude of the biomolecular reaction is measured using a second molecule called a reporter. The reporter molecule signals the extent of the reaction by attaching to the molecules on the microspheres. As both the microspheres and the reporter molecules are color coded, digital signal processing allows the translation of signals into real-time, quantitative data for each reaction. The standard technique is described in U.S. Pat. Nos. 5,736,303 and 6,057,107, herein incorporated by reference.

### Example X

### Identification Techniques

Amplification products may be visualized in order to confirm amplification of the target-gene(s) sequences. One typical visualization method involves staining of a gel with a flourescent dye, such as ethidium bromide or Vistra Green, and visualization under UV light. Alternatively, if the amplification products are integrally labeled with radio- or fluorometrically-labeled nucleotides, the amplification products can be exposed to x-ray film or visualized under the appropriate stimulating spectra following separation.

In one embodiment, visualization is achieved indirectly, using a nucleic acid probe. Following separation of amplification products, a labeled, nucleic acid probe is brought into contact with the amplified products. The probe preferably is conjugated to a chromophore but may be radiolabeled. In another embodiment, the probe is conjugated to a binding partner, such as an antibody or biotin, where the other member of the binding pair carries a detectable moiety. In other embodiments, the probe incorporates a fluorescent dye or label. In yet other embodiments, the probe has a mass label that can be used to detect the molecule amplified. Other embodiments also contemplate the use of TAQMAN and MOLECULAR BEACON probes. In still other embodiments, solid-phase capture methods combined with a standard probe may be used.

The type of label incorporated in DNA amplification products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, primers for the amplification reactions can be labeled with a fluorophore, a chromophore or a radioisotope, or by associated enzymatic reaction. Enzymatic detection involves binding an enzyme to a primer, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If amplification products are separated using a mass spectrometer no label is required because nucleic acids are detected directly.

A number of the above separation platforms can be coupled to achieve separations based on two different properties. For example, some of the PCR primers can be coupled with a moiety that allows affinity capture, while some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

### Example XI

### Kits

The materials and reagents required for the disclosed amplification method may be assembled together in a kit. The kits of the present disclosure generally will include at least the transposome (consists of transposase enzyme and transposon DNA), nucleotides, and DNA polymerase necessary to carry out the claimed method along with primer sets as needed. In a preferred embodiment, the kit will also contain directions for amplifying DNA from DNA samples. Exemplary kits are those suitable for use in amplifying whole genomic DNA. In each case, the kits will preferably have distinct containers for each individual reagent, enzyme or reactant. Each agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions are preferably provided with the kit.

### Example XII

### Embodiments

The present disclosure provides a method a method of genomic nucleic acid amplification including treating genomic DNA in aqueous media with a plurality of dimers of a transposase bound to transposon DNA, wherein the transposon DNA includes a transposase binding site and a specific PCR primer binding site, wherein the plurality of dimers bind to target locations along the double stranded nucleic acid and the transposase cleaves the genomic DNA into a plurality of double stranded genomic DNA fragments representing a genomic DNA fragment library, with each double stranded genomic DNA fragment having the transposon DNA bound to each 5′ end of the double stranded genomic DNA fragment, gap filling a gap between the transposon DNA and the genomic DNA fragment to form a library of double stranded genomic DNA fragment extension products having specific PCR primer binding sites at each end, dividing the aqueous media into a large number of aqueous droplets within an oil phase wherein each aqueous droplet includes no more than one single double stranded genomic DNA fragment and further includes amplification reagents, for each aqueous droplet, amplifying the double stranded genomic DNA fragment therein to create amplicons of the double stranded genomic DNA fragment within the aqueous droplet, wherein amplification takes place in all droplets of the subset, and collecting the amplicons from the aqueous droplets by demulsification of the aqueous droplets.

The present disclosure provides a method of genomic nucleic acid amplification including contacting genomic DNA with a plurality of dimers of a transposase bound to transposon DNA, wherein the transposon DNA includes a transposase binding site, an optional barcode sequence, and a primer binding site, wherein the plurality of dimers bind to target locations along the double stranded nucleic acid and the transposase cleaves the genomic DNA into a plurality of double stranded genomic DNA fragments representing a genomic DNA fragment library, with each double stranded genomic DNA fragment having the transposon DNA bound to each 5′ end of the double stranded genomic DNA fragment, gap filling a gap between the transposon DNA and the genomic DNA fragment to form a library of double stranded genomic DNA fragment extension products having primer binding sites at each end, creating a subset of a plurality of aqueous droplets within an oil phase wherein each aqueous droplet of the subset includes a single double stranded genomic DNA fragment extension product of the library and amplification reagents, for each aqueous droplet of the subset, amplifying the double stranded genomic DNA fragment therein to create amplicons of the double stranded genomic DNA fragment within the aqueous droplet, wherein amplification takes place in all droplets of the subset, and collecting the amplicons from within the aqueous droplets of the subset. According to one aspect, the genomic DNA is whole genomic DNA obtained from a single cell. According to one aspect, the transposase is Tn5 transposase. According to one aspect, the transposon DNA includes a barcode sequence. According to one aspect, the transposon DNA includes a barcode sequence and with the primer binding site being at the 5′ end of the transposon DNA. According to one aspect, the transposon DNA includes a double-stranded 19 bp Tnp binding site and an overhang, wherein the overhang includes a barcode sequence and a primer binding site at the 5′ end of the overhang. According to one aspect, bound transposases are removed from the double stranded fragments before gap filling and extending of the double stranded genomic DNA fragments. According to one aspect, the transposases are Tn5 transposases each complexed with a transposon DNA, wherein the transposon DNA includes a double-stranded 19 bp Tnp binding site and an overhang, wherein the overhang includes a barcode sequence and a primer binding site. According to one aspect, the method further includes the step of sequencing the amplicons collected from within the aqueous droplets of the subset. According to one aspect, the method further includes the step of detecting single nucleotide variations within the amplicons collected from within the aqueous droplets of the subset. According to one aspect, the method further includes the step of detecting copy number variations within the amplicons collected from within the aqueous droplets of the subset. According to one aspect, the method further includes the step of detecting structural variations within the amplicons collected from within the aqueous droplets of the subset. According to one aspect, the genomic DNA is from a prenatal cell. According to one aspect, the genomic DNA is from a cancer cell. According to one aspect, the genomic DNA is from a circulating tumor cell. According to one aspect, the genomic DNA is from a single prenatal cell. According to one aspect, the genomic DNA is from a single cancer cell. According to one aspect, the genomic DNA is from a single circulating tumor cell. According to one aspect, the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library. According to one aspect, the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library and wherein the plurality of aqueous droplets are spontaneously created. According to one aspect, the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library and wherein the plurality of aqueous droplets are created by vigorously mixing the oil phase and the aqueous media. According to one aspect, the subset of the plurality of aqueous droplets within the oil phase are created by combining the oil phase and the aqueous media within a microfluidic chip. According to one aspect, the amplification of the double stranded genomic DNA fragment within each aqueous droplet of the subset is carried out within a microfluidic chip. According to one aspect, the primer binding site is a specific PCR primer binding site. According to one aspect, amplification taking place in all droplets of the subset is PCR amplification using a specific primer sequence.

What is claimed is:

1. A method of genomic nucleic acid amplification comprising
contacting genomic DNA with a plurality of dimers of a transposase bound to transposon DNA, wherein the transposon DNA includes a transposase binding site, an optional barcode sequence, and a primer binding site, wherein the plurality of dimers bind to target locations along the double stranded nucleic acid and the transposase cleaves the genomic DNA into a plurality of double stranded genomic DNA fragments representing a genomic DNA fragment library, with each double stranded genomic DNA fragment having the transposon DNA bound to each 5′ end of the double stranded genomic DNA fragment,
gap filling a gap between the transposon DNA and the genomic DNA fragment to form a library of double stranded genomic DNA fragment extension products having primer binding sites at each end,
creating a subset of a plurality of aqueous droplets within an oil phase wherein each aqueous droplet of the subset includes a single double stranded genomic DNA fragment extension product of the library and amplification reagents,
for each aqueous droplet of the subset, amplifying the double stranded genomic DNA fragment therein to create amplicons of the double stranded genomic DNA fragment within the aqueous droplet, wherein amplification takes place in all droplets of the subset, and
collecting the amplicons from within the aqueous droplets of the subset.

2. The method of claim 1 wherein the genomic DNA is whole genomic DNA obtained from a single cell.

3. The method of claim 1 wherein the transposase is Tn5 transposase, Mu transposase, Tn7 transposase or IS5 transposase.

4. The method of claim 1 wherein the transposon DNA includes a barcode sequence.

5. The method of claim 1 wherein the transposon DNA includes a barcode sequence and with the primer binding site being at the 5′ end of the transposon DNA.

6. The method of claim 1 wherein the transposon DNA includes a double-stranded 19 bp Tnp binding site and an overhang, wherein the overhang includes a barcode sequence and a primer binding site at the 5′ end of the overhang.

7. The method of claim 1 wherein bound transposases are removed from the double stranded fragments before gap filling and extending of the double stranded genomic DNA fragments.

8. The method of claim 1 wherein the transposases are Tn5 transposases each complexed with a transposon DNA, wherein the transposon DNA includes a double-stranded 19 bp Tnp binding site and an overhang, wherein the overhang includes a barcode sequence and a primer binding site.

9. The method of claim 1 further including the step of sequencing the amplicons collected from within the aqueous droplets of the subset.

10. The method of claim 1 further including the step of detecting single nucleotide variations within the amplicons collected from within the aqueous droplets of the subset.

11. The method of claim 1 further including the step of detecting copy number variations within the amplicons collected from within the aqueous droplets of the subset.

12. The method of claim 1 further including the step of detecting structural variations within the amplicons collected from within the aqueous droplets of the subset.

13. The method of claim 1 wherein the genomic DNA is from a prenatal cell.

14. The method of claim 1 wherein the genomic DNA is from a cancer cell.

15. The method of claim 1 wherein the genomic DNA is from a circulating tumor cell.

16. The method of claim 1 wherein the genomic DNA is from a single prenatal cell.

17. The method of claim 1 wherein the genomic DNA is from a single cancer cell.

18. The method of claim 1 wherein the genomic DNA is from a single circulating tumor cell.

19. The method of claim 1 wherein the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library.

20. The method of claim 1 wherein the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library and wherein the plurality of aqueous droplets are spontaneously created.

21. The method of claim 1 wherein the plurality of aqueous droplets within the oil phase are created by combining oil with a volume of aqueous media including the library of double stranded genomic DNA fragment extension products and amplification reagents in a manner to create more droplets than there are double stranded genomic DNA fragment extension products in the library and wherein the plurality of aqueous droplets are created by vigorously mixing the oil phase and the aqueous media.

22. The method of claim 1 wherein the subset of the plurality of aqueous droplets within the oil phase are created by combining the oil phase and the aqueous media within a microfluidic chip.

23. The method of claim 1 wherein amplification of the double stranded genomic DNA fragment within each aqueous droplet of the subset is carried out within a microfluidic chip.

24. The method of claim 1 wherein the primer binding site is a specific PCR primer binding site.

25. The method of claim 1 wherein amplification taking place in all droplets of the subset is PCR amplification using a specific primer sequence.

26. A method of genomic nucleic acid amplification comprising
treating genomic DNA in aqueous media with a plurality of dimers of a transposase bound to transposon DNA, wherein the transposon DNA includes a transposase binding site and a specific PCR primer binding site, wherein the plurality of dimers bind to target locations along the double stranded nucleic acid and the transposase cleaves the genomic DNA into a plurality of double stranded genomic DNA fragments representing a genomic DNA fragment library, with each double stranded genomic DNA fragment having the transposon DNA bound to each 5′ end of the double stranded genomic DNA fragment,
gap filling a gap between the transposon DNA and the genomic DNA fragment to form a library of double stranded genomic DNA fragment extension products having specific PCR primer binding sites at each end,
dividing the aqueous media into a large number of aqueous droplets within an oil phase wherein each aqueous droplet includes no more than one single double stranded genomic DNA fragment and further includes amplification reagents,
for each aqueous droplet, amplifying the double stranded genomic DNA fragment therein to create amplicons of the double stranded genomic DNA fragment within the aqueous droplet, wherein amplification takes place in all droplets of the subset, and
collecting the amplicons from the aqueous droplets by demulsification of the aqueous droplets.
